(19) United States
(12) Patent Application Publication
(10) Pub. No.: US 2014/0358155 A1
(43) Pub. Date: Dec. 4, 2014

(54) INTRAOCULAR LENS PERIPHERAL SURGICAL SYSTEMS

(71) Applicants: Charles DeBoer, Sierra Madre, CA (US); Craig Alan Cable, II, Mission Viejo, CA (US); Ramiro Magalhaes Ribeiro, South Pasadena, CA (US); Matthew McCormick, Yucaipa, CA (US); Sean Caffey, Hawthorne, CA (US); Yu-Chong Tai, Pasadena, CA (US); Mark S. Humayun, Glendale, CA (US)

(72) Inventors: Charles DeBoer, Sierra Madre, CA (US); Craig Alan Cable, II, Mission Viejo, CA (US); Ramiro Magalhaes Ribeiro, South Pasadena, CA (US); Matthew McCormick, Yucaipa, CA (US); Sean Caffey, Hawthorne, CA (US); Yu-Chong Tai, Pasadena, CA (US); Mark S. Humayun, Glendale, CA (US)

(21) Appl. No.: 14/289,193

(22) Filed: May 28, 2014

Related U.S. Application Data
(60) Provisional application No. 61/828,018, filed on May 28, 2013.
(60) Provisional application No. 61/829,607, filed on May 31, 2013.
(60) Provisional application No. 61/862,806, filed on Aug. 6, 2013.
(60) Provisional application No. 61/930,690, filed on Jan. 23, 2014.

(51) Int. Cl. A61F 2/16

(52) U.S. Cl. CPC A61F 2/1662
USPC 606/107

(57) ABSTRACT

Peripheral surgical systems are used for insertion and filling of fluid-filled intraocular lenses, reaccessing and modifying fluid-filled intraocular lenses, and explantation of lenses. Although one peripheral surgical unit may perform all of these functions, in some embodiments different units perform different functions—i.e., each function may be performed by a separate unit, or the functions may be distributed over a smaller number of functional units.

### CROSS-REFERENCE TO RELATED APPLICATIONS

The present application claims priority to, and the benefits of, U.S. Ser. Nos. 61/828,018 (filed on May 28, 2013), 61/829,607 (filed on May 31, 2013), 61/862,806 (filed on Aug. 6, 2013), and 61/930,690 (filed on Jan. 23, 2014). The entire disclosures of these priority documents are hereby incorporated by reference.

### FIELD OF THE INVENTION

In various embodiments, the present invention relates generally to implantable intraocular lenses and, more specifically, to peripheral surgical systems relating to fluid-filled intraocular lenses.

### BACKGROUND

The crystalline lens of a human's eye refracts and focuses light onto the retina. Normally the lens is clear, but it can become opaque (i.e., when developing a cataract) due to aging, trauma, inflammation, metabolic or nutritional disorders, or radiation. While some lens opacities are small and require no treatment, others may be large enough to block significant fractions of light and obstruct vision.

Conventionally, cataract treatment involves surgically removing the opaque lens matrix from the lens capsule using, for example, phacoemulsification and/or a femtosecond laser through a small incision in the periphery of the patient's cornea. An artificial intraocular lens (IOL) can then be implanted in a lens capsule bag (the so-called “in-the-bag implantation”) to replace the crystalline lens. Generally, IOLs are made of a foldable material, such as silicone or acrylics, for minimizing the incision size and required stitches and, as a result, the patient's recovery time. The most commonly used IOLs are single-element lenses (or monofocal IOLs) that provide a single focal distance; the selected focal length typically affords fairly good distance vision. However, because the focal distance is not adjustable following implantation of the IOL, patients implanted with monofocal IOLs can no longer focus on objects at a close distance (e.g., less than 25 cm); this results in poor visual acuity at close distances.

The insertion system for traditional IOLs typically involves an insertion device body and a small-diameter insertion tube through which the IOL travels. The insertion tube is placed into the surgical incision in the eye and the IOL is pushed from the insertion device body through the tube and inserted into the eye. Normally a viscoelastic, such as a hyaluronic acid or equivalent, is used to lubricate the lens as it passes through the insertion tube. After insertion, the IOL unfolds and is positioned into the correct anatomical location, most often the lens capsule.

Recently, liquid-filled intraocular lenses have been developed; these may be inserted into the eye and then filled. Advantages of this design include the ability to deploy through a small incision, following which the lens is inflated in situ. A small insertion diameter reduces post-operative healing times, allows the surgeon to avoid sutures for closing the incision, and reduces post-operative astigmatism. Therefore, incisions less than 3 mm, and preferably less than 2 mm, are desired by operating personnel for better surgical outcomes. In addition, certain liquid-filled intraocular lens designs can be adjustable after implantation to ensure accurate vision by refractive corrections through adjustment of the filling medium inside the lens. When made flexible, the fluid-filled lenses can provide adjustable focal distances (or accommodation), relying on the natural focusing ability of the eye (e.g., using contractions of ciliary muscles). Unlike traditional intraocular lenses, which are not filled after insertion, liquid lenses designed to be deployed in a semi-deflated or completely deflated state (both cases referred to herein as a “deflated state”) are both deployed into the eye and then inflated after deployment. Specialized insertion and filling systems are, therefore, generally required to implant these lenses. Additionally, these lenses can have the fluid contents adjusted after implantation. Therefore, there is a need for tools to access the fluidic contents of the fluid-filled IOL and to adjust the contents of the IOL before, during, and after implantation.

### SUMMARY

Peripheral surgical systems in accordance herewith are used for insertion and filling of fluid-filled intraocular lenses, reaccessing and modifying the lenses, and explantation of the lenses. Although one peripheral surgical unit may perform all of these functions, in some embodiments different units perform different functions—i.e., each function may be performed by a separate unit, or the functions may be distributed over a smaller number of functional units. The invention may also be used as a peripheral surgical system for other fluid-filled implantable devices such as a scleral buckle or breast implant.

In one aspect, the present invention relates to an intraocular lens insertion and filling system. Various embodiments contain a fluidic line in fluidic continuity with a deflated intraocular lens and an insertion system for deploying the intraocular lens into the eye. The fluidic system is used to fill the lens with a fluid after deployment of the lens into the eye. As used herein, the term “fluid” generally refers to a liquid, but in some instances may refer to or encompass a gas and/or a solute. For example, gases would not be suitable for implants as barometric changes would cause unwanted changes in accommodation.

The fluidics system may comprise an infusion pump, although an aspiration pump may be used alternatively or in addition. The infusion pump is responsible for dispensing fluid into a fluid-filled intraocular lens. In one embodiment, the infusion pump consists of or comprises a syringe pump capable of dispensing accurate volumes of fluid. This is especially suited for viscous fluids, such as silicone oils, where high pressures may be required in order to dispense at an adequate rate. Furthermore, a syringe pump reduces pressure surging that may occur with other pump technologies.

When present, the aspiration pump is responsible for removing media from the IOL. Suitable aspiration pumps include but are not limited to gear pumps, peristaltic pumps, venturi pumps, and syringe pumps. Certain pumps may be placed directly in line with the aspiration line without contaminating it. For example, a peristaltic pump can have the tubing from the aspiration side of the pump attached to it. Other pumps attach to a cassette, which is in fluidic contact with the aspiration line. Examples of this include pumps that operate with air, e.g., venturi pumps that are attached to a vacuum reservoir. The pump is used to evacuate air from the reservoir, which then drives fluid into the reservoir. However, fluid never contacts the pump in this implementation.

In certain embodiments, the infusion pump and aspiration pump have distinct fluidic lines connected to the handpiece. In one embodiment, two distinct lines carry infusion and aspiration, respectively. In this configuration, the handpiece tip utilizes two cannulas, configured either side-by-side or concentrically. One cannula is used for injection of fluid into the IOL, while the other aspirates. Infusion and aspiration can occur simultaneously. This approach is advantageous for, e.g., fluid exchange of the IOL. One specific use of fluid exchange is removing fluid of one refractive index and replacing it with fluid of another refractive index. In certain embodiments, the refractive index of the lens filling fluid is monitored during lens fluid exchange and used to determine the amount of fluid to exchange. It is preferable to make the aspiration cannula larger than the infusion cannula because aspiration is limited to a maximum vacuum of one atmosphere, whereas infusion can occur at much larger pressure differentials.

In another embodiment, the aspiration line and the infusion line meet in a valve and are carried to the tip of the device through a single line. The tip typically has a single cannula. When infusion is active, it occurs through the tip of the device. When aspiration is active, the valve is in the opposite position, and fluid from the tip is aspirated. This provides the largest total area for both infusion and aspiration for a specific tip size. In a third position, the infusion and aspiration lines are in fluidic connection. This configuration is not limiting, of course, and other modes of switching between lines can be used—e.g., closing lines separately and remotely.

The aspiration line in this embodiment of the invention can be used to prime the line and remove air bubbles therefrom. The aspiration line and infusion line may meet in a valve or y-connection close to the distal end of the tip. With the aspiration and infusion line in fluidic connection, vacuum is applied to the aspiration line during fluid infusion. Infused fluid follows a path from the infusion side of the injector and then directly to the aspiration line, never moving to the most distal end of the tip. Therefore, no fluid travels out of the tip, keeping it clean from fluid residue while allowing all lines to be primed and purged of air. Maintaining a clean injector tip is desirable when accessing a valve of a liquid-filled IOL to prevent any liquid from contacting the external surface of the IOL. In addition, this is desirable when the lens is in fluidic contact with the tip. The lens can be put into fluidic contact with air in the lines, e.g., before attaching the fluidic system. Then the system is primed with the lens directly connected to the injection tip. For example, the lens may be mounted to the injection tip before the filling fluid is connected to the injector, following which the filling fluid is connected to the injector; after connection of the filling fluid, the lines are primed by infusion fluid through the infusion line and aspiration through the aspiration line. Although vacuum is discussed as being used with the aspiration lines, this is not required. If the aspiration line has low fluidic resistance relative to other parts of the system, or if a valve closes the distal end of the tip, no vacuum is required to prime the line. In addition, the line may end in a reservoir in the handpiece to allow collection of the fluid. The reservoir may have a semipermeable membrane to allow fluid to fill the reservoir while air freely passes out of the reservoir.

In some embodiments, a selective filter, such as a degassing or debubbling filter, is used to remove air from the liquid and the lines. The selective filter acts to allow air, but not the fluid, to pass through. During priming of the lines and infusion of the fluid, the air and air bubbles are drawn from the lines through this selective filter. As an example, a semipermeable membrane tube may be used as a portion of the infusion line. Vacuum is applied externally to the semipermeable membrane tube. As air or the fluid passes through that portion of the filling tube, the external vacuum removes the air from the line. Alternatively or in addition, an air-capture device, such as an out-pocket in the infusion line, may be used to capture air bubbles as they pass through the infusion line, preventing air bubbles from entering the lens.

In various embodiments, a single pump is used for both aspiration and infusion through a single or multiple cannulas.

The tip of the handpiece may comprise one or more cannulas used to access the internal contents of the liquid-filled IOL. In one embodiment, the tip includes or consists of a blunt cannula, with a thin-walled, reduced-diameter polymer at the distal end. The polymer is selected to retain enough rigidity to access the lens, but a blunt end prevents damage to the lens walls. Suitable polymers include, but are not limited to, polyimide, TEFLON, PEEK, polyester, NYLON, polyethylene, and ABS.

In certain embodiments of the invention, the infusion and aspiration system is used to monitor the volume of fluid infused into or aspirated from the intraocular lens. Alternatively or in addition, the pressure inside the lens may be monitored. The refractive index of the filling fluid may also (or alternatively) be monitored, e.g., by an inline refractometer. Monitoring filling or aspiration, pressure inside the lens, or the refractive index of filling fluid can be used to determine the amount of lens fill, the amount of fluid to exchange, refractive properties of exchange fluid, and optical properties of the lens. Therefore, this approach can be used to determine the appropriate refractive power of the implanted intraocular lens.

In certain embodiments, the IOL is loaded by inserting a sharp point into a valved portion of the lens or a polymeric membrane in the IOL. Then a cannula is inserted into the valved portion/polymeric membrane with the sharp point over the sharp point, similar to a trocar cannula insertion, or after the sharp point has been removed. If used in the manner of a trocar cannula insertion, the sharp point is removed after insertion of the cannula.

In a representative example of use, first the IOL is accessed via a sealing portion thereof with a sharp point, such as a sharpened nitinol wire protruding through the tip of the insertion and filling system. Next, the cannulated tip of the injection system is inserted through the sealing portion of the IOL. The nitinol wire is removed from the injector and the lens is tested for sealing using pressure, flow, optical, or visual monitoring of the lens. If the lens passes the sealing test, it is deflated and drawn into the insertion tube. The fluidics lines are attached to the lens. In certain embodiments, the lines are primed before attachment to the insertion system. In other embodiments, the lines are primed after attachment to the insertion system, while the lens is attached to the insertion and filling system.

In a representative system embodiment, an intraocular lens insertion and filling system according to the invention comprises a fluidic system in connection with the inside of an intraocular lens; the fluidic system is capable of filling or removing fluid from the intraocular lens after implantation into the eye. The intraocular lens is deployed from the insertion tip using a mechanical and/or fluidic force and is subsequently inflated by the insertion and filling system. The system may be configured to measure the pressure of the intraocular lens; the fluid flow and volume injected into or removed from the intraocular lens; and/or the refractive index of the fluid inside the intraocular lens. In some embodiments, a plunger is used to provide a seal around the lumen of the insertion system and insert the lens into the eye using a fluidic force created by the seal of the plunger.

In certain embodiments, a sheath wraps around the lens during loading and/or insertion of the lens. A mechanical gripping mechanism comprising two or more members may be used to draw the lens into and expel the lens from the insertion system. For example, the gripping system may be used to re-access a sealing portion of the IOL after implantation of the intraocular lens.

In some embodiments, the insertion tube is translucent or clear for visualization of the loaded lens. The intraocular lens may be monitored for leakage by one or more of visual detection, optical detection, pressure monitoring, or flow monitoring.

In another aspect, the invention pertains to an intraocular lens-adjustment system for accessing an interior of an intraocular lens following implantation thereof. In various embodiments, the system comprises an access tip configured for mechanical interface with a valve of the lens via an exterior surface thereof, the access tip, when engaged with the valve, forming a fluidic seal therewith; one or more reservoirs used to store a fluid; and one or more fluidic lines for conducting the stored fluid between the reservoir and the access tip.

The system may further comprise a handpiece attached to the fluidics line and facilitating movement of the access tip relative to the intraocular lens valve. For example, the handpiece may comprise means for controlling a flow of fluid between the reservoir and the access tip. In some embodiments, the fluidics line has minimal wall compliance and is capable of carrying fluids at pressures over 10 PSI.

In various embodiments, the system further comprises a plurality of sensors and a controller connected thereto, the sensors measuring fluid flow in the one or more fluidic lines, a refractive state of the lens, and an internal pressure of the lens, the controller being responsive to the sensors and to a geometric shape of the lens. A portion of at least one fluidics line may have a diameter less than 4 mm to allow reaccess to a previous main conical incision without widening the incision. The access tip may have a diameter less than 3 mm to allow self-sealing of a valve.

In a typical implementation, the system comprises at least one mechanical pump for driving fluid between the reservoir and the access tip. The system may include a metering device to monitor the fluid added or removed from the lens. In some embodiments, a flow sensor is located in proximity to the access tip to account for capacitive changes in the fluid or cavitation. A pressure sensor, if present, may be extendable past the access tip to directly monitor the pressure inside the lens. Alternatively or in addition, a pressure sensor may measure pressure outside the lens.

In various embodiments, the access tip comprises a locking feature for mechanically engaging the valve. For example, the locking feature may be a tether, a vacuum, a twist-lock, and/or a gripper.

In another aspect, the invention relates to an intraocular lens explantation system. In various embodiments, the system comprises an aspiration pump; a conduit fluidly coupled to the pump, the conduit having a distal end; an access member at the distal end of the conduit, the access member being configured to establish fluid communication between the pump and an interior of the lens, and including (i) an opening, (ii) a peripheral contact surface surrounding the opening, (iii) a passage fluidly coupling the opening to a lumen of the conduit, and a gripping member extending axially through the passage and beyond the opening, the gripping member including a mechanical feature for gripping an interior wall of the lens with the peripheral contact surface against an outer surface of the lens.

In some embodiments, the gripping member is retractable through the passage to pull the lens therein. The mechanical feature may be, for example, a barb or a pair of grippers in a forceps configuration.

Still another aspect of the invention relates to an intraocular lens explantation system. In various embodiments, the system comprises an aspiration pump; a conduit fluidly coupled to the pump, the conduit having a distal end; an access member at the distal end of the conduit, the access member establishing fluid communication between the pump and an interior of the lens and including an opening, a peripheral contact surface surrounding the opening, a passage fluidly coupling the opening to a lumen of the conduit, and a cutting member for cutting the lens to establish fluid communication between an interior of the lens and the pump.

In some embodiments, the the cutting member is disposed within the passage, suction created by the pump causing contact between the cutting member and the lens. The cutting member may be disposed telescopically within the passage and have a blade surrounding a central bore, the central bore being in fluid communication with the pump to apply suction to the lens. The cutting member may be configured for axial, rotary or reciprocating movement. In some embodiments, the cutting member is a laser.

Another representative system embodiment comprises a fluidic system in fluid communication with the inside of an intraocular lens and capable of filling the intraocular lens after implantation into the eye. A second fluidic system is used to infuse fluid through the insertion tip and assist in deploying the intraocular lens into the eye, and the intraocular lens may be deployed from the insertion tip using a combination of mechanical and fluidic force. The lens is subsequently inflated by the insertion and filling system.

Yet another representative system embodiment includes a fluidic system in communication with the inside of an intraocular lens and capable of filling the intraocular lens after implantation into the eye. The system also includes one or more of an infusion system used to infuse fluid into the eye before, during, or after implantation of the intraocular lens; or an aspiration system used to infuse fluid into the eye before, during, or after implantation of the intraocular lens

Still another representative system embodiment comprises a fluidic system in communication with the inside of an intraocular lens and capable of filling the intraocular lens after implantation into the eye. The intraocular lens is deployed from the insertion tip and is subsequently inflated by the insertion and filling system. The system is configured to permit infusion and aspiration through a single or multiple lumens.

Yet another representative system embodiment comprises a fluidic system in communication with the inside of an intraocular lens and capable of filling the intraocular lens after implantation into the eye. The system is configured such that, after insertion of the intraocular lens and insertion tip, the insertion tip retracts from the intraocular lens and the intraocular lens is inflated.

Another representative intraocular lens insertion and filling system in accordance with the invention comprises a fluidic system in communication with the inside of an intraocular lens and capable of filling the intraocular lens after implantation into the eye. In this embodiment, the fluidic system comprises three separate fluidic lines: an infusion line, an aspiration or bleed off line, and a tip used to access the IOL. These three separate fluidic lines may connect by means of a y-connector or valve. During system priming, air and fluid pass from the infusion line to the aspiration or bleed-off line, and upon inflation of the IOL fluid passes from the infusion line to the aspiration line.

A representative method in accordance with the invention for preparing an IOL for implantation comprises inserting a fluidic line in the IOL, inflating the IOL using air or fluid, and inspecting the IOL for leakage visually, optically, using pressure, and/or fluid flow. After the lens has been deemed not to leak, the IOL may be deflated and drawn into tube for insertion into the eye.

In another representative method, fluidic continuity is provided between the intraocular lens and a filling system, the intraocular lens is deployed into the eye using a mechanical and/or fluidic force, and the intraocular lens is inflated. For example, the intraocular lens and an insertion tip may be inserted into the eye, the insertion tip may be retracted around the intraocular lens, and the intraocular lens may be inflated.

In aspects, the invention is directed toward re-access to a fluid-filled intraocular lens through a valve or re-access port that may comprise or consist of a fluidic connection coupling the fluid-filled intraocular lens with either a valve or self-sealing medium in a tube. This re-access is performed to either inflate, deflate, or exchange fluid. When referring to inflating a fluid-filled intraocular lens, this could substantially refer to the process of injecting additional fluid into the lens which already contains a fluid, and injecting a soluble material or a non-soluble material or a pharmaceutical drug into the preexisting fluid. The primary purpose of injecting fluid that is identical in composition to that of fluid already existing in a fluid-filled intraocular lens is to change the volume of the lens. This then changes the curvatures of radius on either the anterior, posterior or both curvatures of the lens according to the design of the lens. This will then change the base power of the lens, thereby the index of refraction of the cornea. Base power change can similarly be accomplished by removing fluid from the fluid-filled intraocular lens.

In other embodiments, the anterior and posterior curvatures of the lens are not changed during filling but different properties of the lens are. One embodiment allows for changes of the intraocular lens size, allowing a better conformal fit between the intraocular lens and the surrounding lens capsule. In yet another embodiment in which the anterior and posterior curvatures are not changed, a fluid of different refractive index is injected, thereby altering the refractive index of the fluid-filled intraocular lens. A soluble example would be injecting a high concentration sugar water into a water based filled lens. Because refractive index is altered by the material compositions and may be altered by dopants (i.e. sugar concentration), a higher sugar concentration can be used to increase the refractive index of a filling fluid. Many other dopants sized below the scattering coefficient may be substituted. Additional other factors including pressure of the liquid, temperature, and frequency of light further alter the refractive index.

In another embodiment, crosslinking agents are injected into an uncured or partially cured silicone filled lens. During the curing process of the silicone (i.e., baking, time, UV exposure), crosslinking occurs and the refractive properties of the silicone molecule change, thereby altering refractive index. In other embodiments, different crosslinking agents compatible with the curing methods of alternative materials besides silicone may be used. Specific examples include hydrogel, acrylic, phenyl-substituted silicone, or fluorosilicone. In other embodiments, the fluid injected into the lens is a chemically modified species to crosslink or chemically bond with the existing internal contents of the lens. As an example, phenyl-substituted silicones have a higher refractive index than non-phenyl-substituted silicones. The refractive index is proportional to the amount of phenyl-substituted entities in the silicone. Therefore, by taking a low level of phenyl-substituted silicone and adding monomers with phenyl-substitution into the internal contents of the lens, the refractive index can be increased. Likewise, by crosslinking in an unsubstituted, or low-level substituted silicone with an existing phenyl-substituted silicone, the refractive index can be decreased. Crosslinking may occur over a long period of time, longer than 6 hours, and in some embodiments longer than three months. In certain embodiments, crosslinking has been mostly completed by 90 days, thereby allowing the refractive properties of the lens to be adjusted up to 90 days by altering the inner composition until fully cured. In other embodiments, crosslinking is never complete, and a light crosslinking yields a gel that is capable of being modified throughout the life of the implant.

In other embodiments, insoluble liquid is injected to inflate the lens and increase the volume of the lens so it can either reshape the tissue around the lens or break existing bonds of tissue to the lens. This can be done by injecting air into the fluid-filled intraocular lens. The air can then diffuse out through the membrane of the lens. Other reasons for injecting a soluble or non-soluble into a fluid-filled intraocular lens is to reduce the amount of ultraviolet light that passes through the lens. A pharmaceutical drug can also be injected into the fluid-filled intraocular lens for extended drug delivery. In certain embodiments of the invention the pharmaceutical is injected into the lens periodically to ensure proper levels of intraocular drug are maintained in the eye. In certain embodiments of the invention there is a separate chamber in the fluid-filled intraocular lens, into which the drug can be injected into and diffuse out into the eye over time, without altering the refractive index of the lens.

The tip of the re-access tool, which contains the component that accesses the fluid within the fluid-filled intraocular lens, depends on the valve or re-access port configuration which it is accessing. In one form the tip pierces the valve and then the valve self-seals after removal of the tip. This tip configuration would preferably have a sharp point to help pierce through the valve while having non-coring properties to minimize valve material removal. Another embodiment would be a semi-blunt or blunt tip that would be guided into a preexisting passage way. An example of a semi-blunt tip has a bevel like a sharp tip, however, instead of terminating at a sharp point, the tip of the bevel is manufactured to have a blunt end. This blunt end is designed to allow access to the valve while minimizing damage to the valve and surrounding intraocular lens, even when misguided by the user. This design mitigates the need to protect the remaining lens from a sharp tip to avoid damage or rupture to the intraocular lens. For example, the fluid-filled intraocular lens may be created in thinner embodiments, thereby altering the flexibility, refractive index, and accommodative properties, with minimal risk of rupture by instruments. Examples of the valve design include, but are not limited to a self-sealing hole, check valve, flap valve, or a tube with a valve or self-sealing medium.

Many of these re-access tool embodiments will benefit from a mechanism of alignment to align the tip with the access point. Alignment may be created in various ways. In one embodiment, there are one or more tubes. One tube pulls a vacuum to help grab the valve or tube. This configuration can be created by having concentric tubes, side-by-side tubes or some pre-designed shape that would be characteristic to the access point that the vacuum can hold on to in a certain orientation to line up the access tip to deliver or remove fluid. These redundant tubes may be multiple use, or single use in which case they may be sealed and removed after use.

The re-access tool in a broader sense may comprise or consist of an access tip, connected to a fluidic line or lines, which connects to a console that can have one or more fluid reservoirs for infusion into the lens, a vacuum mechanism for removing fluid from the intraocular lens or both. The filling process can be controlled by a foot pedal switch controlled by the surgeon to allow them to have both hands free to manipulate the tool and the fluid-filled intraocular lens. The switch can also be located on the tool itself and activated by the finger of the surgeon. The amount of fluid injected or removed fluid will is monitored or metered in certain embodiments. This can be done with a flow sensor located on the fluidic line closer to the access tip. The closer to the distal end of the re-access tool, the more accurate the flow sensor. This is because the lines throughout the tool are subject to flexing, even minute amounts during infusion. This causes a capacitive ability of the lines. Therefore, flow from the infusion reservoir can be higher than flow out of the access tip during intraocular lens filling. Therefore, measurements at the proximal end of the line will overestimate the total flow into the intraocular lens as a certain amount of flow. During aspiration of intraocular lens contents, small bubbles in the airline can cavitate. This leads to fluid proximal to the console to be susceptible to erroneously higher aspiration levels than the actual fluid leaving the intraocular lens. For both situations, a flow sensor proximal to the lens is desired for high accuracy flow monitoring. Flow sensors, such as, but not limited to, those based upon thermal effects, time-of-flight, and/or pressure, may be used for monitoring/metering purposes.

The flow sensor can also accurately measure the amount of fluid coming in and out of the fluid-filled intraocular lens. In embodiments of injecting the fluid with a fluidic line that does not have compliance, a flow sensor may not be necessary as a syringe or some kind of accurate dispensing technique can be used to accurately inject fluid. Furthermore, the filling can be controlled by measuring the power of the lens within the patient while injecting or removing fluid. This measurement can then be used as real time feedback to a console that can then control the amount of fluid being injected or removed from the fluid-filled intraocular lens.

Other feedback mechanisms to control fluid infusion include monitoring the overall refractive power of the lens during lens adjustment, monitoring aberration of the lens and/or of the eye during lens adjustment, monitoring refractive index of the filling fluid, and monitoring pressure inside the lens.

In certain embodiments, fluid is altered to change the overall refractive state of the intraocular lens to achieve emmetropia. In other embodiments, lens aberrations, such as Zernicke coefficients, are monitored and adjusted to alter the overall refractive state of the lens as well as aberration of the intraocular lens. As a simple example, the aberration of the implanted lens is adjusted to reduce overall astigmatism of the eye, as in the case of an astigmatic cornea. In other embodiments, spherical aberration is adjusted and possibly increased to increase depth of field of the implanted lens. In other embodiments, aberrations are reduced to increase overall visual acuity. This may occur through a single access valve in the intraocular lens, or multiple valves in the intraocular lens, these valves accessing separate portions or reservoirs of the intraocular lens. These separate portions of the intraocular lens are used to adjust the aberration of the lens as well as power of the lens. In the simplest form, one chamber is used for overall dioptric power of the lens, while a second chamber is used to adjust toricity of the intraocular lens to correct for astigmatism. The re-access tool may then be used to access one or both of the chambers. For example, it may be used to post-operatively adjust the toricity of an implanted intraocular lens for better astigmatic correction. This is important in the case of astigmatism induced by the surgical implantation process of the lens itself, which is difficult to predict. In another example, the re-access tip is used to increase spherical aberration to increase overall depth of field of an implanted intraocular lens. In yet another example, the re-access tool is used to adjust the lens based on unexpected corneal aberration post-operatively. The implanted IOL is adjusted to correct for aberration of the cornea to reduce overall aberration of the cornea-lens optical system of the eye.

Various aspects of the present invention relate to intraocular lens explantation, i.e., removal of liquid-filled IOLs from the eye. Explantation occurs by first removing the fluid from the liquid-filled IOL and then removing the lens in a deflated state. The advantage of this technique is that after removal of fluid, the deflated IOL has a small profile, allowing it to be removed through small incisions. More specifically, removal of the lens with incisions under 3 mm, and in some embodiments of the invention under 1 mm, is possible.

In one embodiment of the invention, a portion of the explantation tool retains the lens using suction. Once the lens is engaged to the explantation tool, a second portion of the tool is used to access the internal contents of the lens, e.g., through a special area of the lens such as a valve or through the wall of the lens. In one implementation a specialized hook is used to enter the lens and cause leakage to the outer member, where the internal liquid is aspirated out of the lens. In other implementations, no gripping tool is used; instead, a hollow cannulated tool is used to access the internal contents of the lens and aspirate the liquid. For example, the cannulated tool may have a sharp end to assist in accessing the liquid-filled intraocular lens. Alternatively, the cannulated tool may have a barb, hook, or other device for mechanically retaining the lens after insertion into the liquid-filled intraocular lens.

In certain embodiments the deflated lens is drawn into the explantation tool for removal from the eye. In other embodiments the deflated lens is removed using a separate portion of the explantation system, which individually grasps and removes the deflated lens. This individual portion of the explantation system may have an aspiration or infusion aspiration component that is used to assist in gripping the lens, maintaining pressure in the anterior chamber of the eye, and in removing any residual liquid from the intraocular lens. Some implementations of the invention use a fluid exchange in the IOL before deflating the IOL and removing it. Aspiration comes from one portion of the explantation system while infusion is applied through the same portion of the IOL or from a separate portion of the lens.

In certain implementations a specific tool is used to open an aperture in the IOL and then aspirate liquid coming from the IOL. Other embodiments aspirate the intraocular lens by first using cautery, laser, ultrasonic power, or mechanical cutting to open an aperture in the device and then aspirate the contents of the intraocular lens.

One implementation of the invention uses a separate line to infuse fluid, such as BSS, viscoelastic, or air into the lens capsule while the lens is being deflated. This technique maintains the natural lens capsular shape, facilitating IOL removal from the lens capsule and subsequent IOL “in the bag” injection with a replacement IOL.

In certain aspects, therefore, the invention pertains to an intraocular lens explantation system. In various embodiments, the system comprises a portion that retains an intraocular lens using a mechanical, suction, or combination of mechanical and suction force to hold the lens, and a portion that accesses the internal contents of a fluid-filled intraocular lens; this latter portion removes or facilitates removal of the contents of the lens before lens removal from the eye. The portion that accesses the internal contents of the IOL may, for example, comprise or consist of a hooked or barbed member, and may be used to mechanically retain the lens against the retention portion of the explantation tool.

The portion that accesses the internal contents of the IOL may alternatively comprise or consist of a cannulated tool that aspirates the contents of the lens while the lens is held by the gripping portion of the explantation tool. The portion that accesses the internal contents of the IOL may comprise or consist of an aspiration infusion portion that aspirates the contents of the lens and infuses a second fluid into the lens in order to fluidically exchange the internal contents of the lens with another fluid. After fluid exchange the lens is evacuated and drawn out of the eye. In still other embodiments, the retention portion may also aspirate fluid from the lens.

The explantation tool may have a feature to draw in the intraocular lens for removal thereof from the eye. A second independent portion of the explantation system, such as a forceps or other gripping member, may be designed specifically to interact and remove the deflated lens.

In another aspect, the invention relates to an intraocular lens explantation system comprising or consisting of two independent components. The first component is an intraocular lens gripper that uses a mechanical, suction, or combination of mechanical and suction force to hold the lens, and also accesses the internal contents of a fluid-filled intraocular lens in order to remove the contents of the lens before lens removal from the eye. The second component accesses a separate portion of the lens to infuse another fluid therein and/or to aspirate fluid from the lens.

An intraocular lens explantation system in accordance with the invention may comprises a tip used to open an aperture in the lens and allow fluid to escape while a second portion of the tip aspirates the fluid from the lens. A portion of the explantation tool may provide for infusion as well as aspiration.

An intraocular lens explantation system in accordance with the invention may comprise a portion that accesses the lens to deflate the lens while a second portion infuses fluid or viscoelastic into the lens capsule while the lens is deflated.

An intraocular lens explantation system in accordance with the invention may have an ultrasonically powered tip used to open an aperture in the side of the liquid-filled intraocular lens and aspirate the lens contents; the ultrasonically powered tip may have aspiration and infusion capability. In some embodiments, the tip contains a sharp portion to assist in rupturing the wall of the liquid-filled intraocular lens.

An intraocular lens explantation system in accordance with the invention may have a cautery tip to open an aperture in a liquid-filled intraocular lens, an aspiration portion to allow fluid from the IOL to be aspirated, and an optional infusion portion.

An intraocular lens explantation system in accordance with the invention may have a laser to open an aperture in a liquid-filled intraocular lens, an aspiration portion to allow fluid from the IOL to be aspirated, and an optional infusion portion. The laser may, for example, be endoscopically operated.

An intraocular lens explantation system in accordance with the invention may have means for cutting the edge of the liquid-filled intraocular lens and aspirating the contents of the intraocular lens. The cutting means may comprise or consist of a cutting tube telescopically received in an outer tube and having a cutting port on the distal end, with suction applied to the cutting port through the center of the inner cutting blade. The cutting tube may cut using one or a combination of reciprocating axial motion, reciprocating rotary motion, or rotary motion.

An intraocular lens explantation system in accordance with the invention may have a portion that accesses the internal contents of a fluid-filled intraocular lens, removing the contents of the lens before lens removal from the eye.

In another aspect, the invention relates to a method of explanting a fluid-filled intraocular lens. In various embodiments, the method consists of or comprises partially or fully emptying the intraocular lens and then removing the lens from the eye, either with the same tool used to empty the lens or a different tool.

A method of explanting a fluid-filled intraocular lens in accordance with the invention may comprise or consist of first exchanging the fluid in the intraocular lens with a second fluid, then partially or fully emptying the intraocular lens, and then removing the lens from the eye, either with the same tool used to empty the lens or a secondary tool. The fluid may be exchanged by means of a single access point in the lens. In some embodiments, the fluid is exchanged using one tool to remove fluid from the lens and a second tool to inflate the lens with a second fluid.

Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology. The term “substantially” or “approximately” means ±10% (e.g., by weight or by volume), and in some embodiments, ±5%.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIGS. 1A and 1B depict the IOL insertion and filling system.

FIG. 2A and FIG. 2B depict the insertion and filling system with a sealing member to deploy the IOL.

FIG. 3A and FIG. 3B depict an implementation of this invention with a protective sheath to assist in deploying the IOL.

FIG. 4A and FIG. 4B depict an implementation with a mechanical gripping mechanism used to fold and deploy the lens.

FIG. 5 depicts an implementation with a fluidic line used to fluidically push the IOL out of the injector.

FIG. 6 depicts the access tip that is a dual cannula providing both infusion and aspiration.

FIG. 7 depicts the insertion and filling system with a separate infusion line and aspiration line attached to the access tip through a y-connector or valve.

FIG. 8 depicts the insertion and filling system with a debubbling filter used with the injection tip.

FIG. 9A-F depict the insertion and filling system with a specific method of checking the lens for leakage after insertion onto the injection and filling system.

FIG. 10 depicts the fully evacuated IOL fluidically connected to the access tip extending out of the insertion tube.

FIG. 11 illustrates a fluid-filled intraocular lens being accessed by an embodiment of a re-access tool.

FIG. 12 illustrates various embodiments of the access tip of the re-access tool.

FIG. 13 illustrates a dual-lumen access tip.

FIG. 14 illustrates various feedback mechanisms incorporated into the re-access tool.

FIG. 15 illustrates an explantation system interacting with an implanted lens.

FIG. 16 illustrates a view of the explantation system interacting with the lens.

FIG. 17 illustrates the deflated IOL in the explantation tool.

FIG. 18 illustrates an embodiment of the invention with a bimanual explantation tool.

FIG. 19 illustrates an explantation tool with a sharp portion that is used to open an aperture in the IOL before aspiration of the IOL contents.

FIG. 20 illustrates an implementation of the invention where the explantation system consists of a cutting tool used to cut a portion of the lens and aspirate the lens and filling fluid.

### DETAILED DESCRIPTION

The peripheral surgical systems described below are used for insertion and filling of fluid-filled intraocular lenses, reaccessing and modifying the fluid-filled intraocular lens, and explantation of the lens. Although one peripheral surgical unit may perform all of these features associated with the surgical manipulation of the fluid-filled intraocular lens, many different units may perform each separate functional feature. The invention may also be used as a peripheral surgical system for other fluid-filled implantable devices such as a scleral buckle or breast implant.

### 1. Insertion/Filling

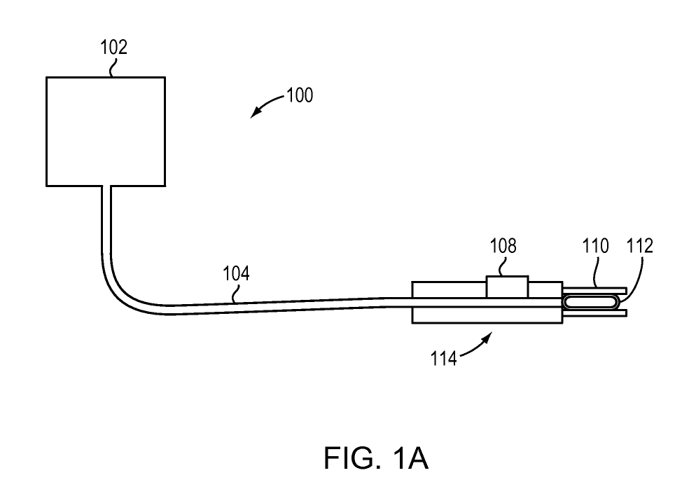

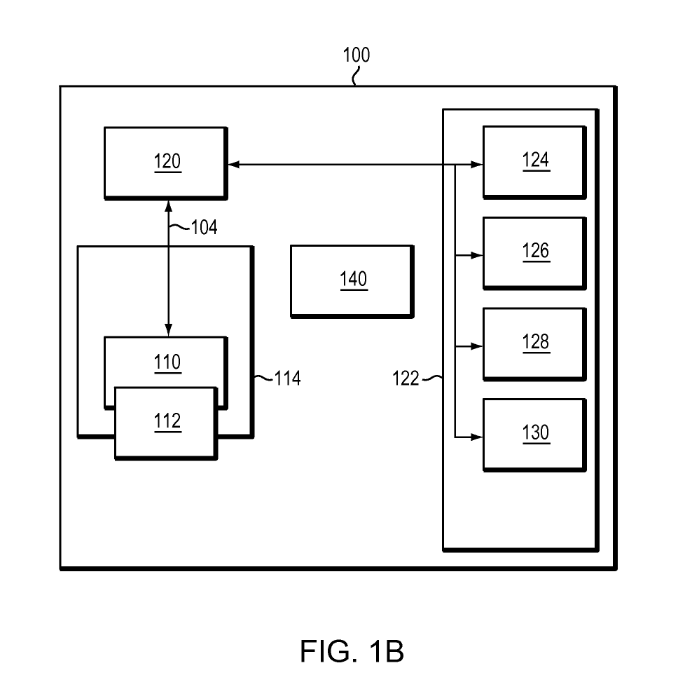

Refer first to FIG. 1A, which depicts a representative IOL insertion and filling system 100. Fluidics line 104 connects the fluidics system 102 to an intraocular lens 112. Intraocular lens 112 is loaded into an insertion tube 110. During implantation, the insertion tube is inserted into the eye through a small incision. Then the intraocular lens 112 in pushed out of the insertion tube 110 into the correct location in the eye. The insertion tube 110 may be configured to be clear or translucent in order for the surgeon to visually inspect the lens during loading, while it is loaded, or during insertion. In FIG. 1A a slider 108 is used to deploy the IOL 112 by mechanically advancing the fluidics line 104 relative to the handpiece 114. However, this is not meant to be limiting and other configurations known to those skilled in the art can be used, including devices such as a lever, ball screw, switch or an automated deployment through an actuator 120 such as pneumatic, motor, or solenoid actuation. Alternatively, any known approach to pump fluid may be utilized. Combinations of one or more actuators 120 may be used in parallel such as one pneumatic pump and one vacuum pump. After filling, the lens is too large to withdraw back into the insertion tube 110, so simple retraction of the fluidics line 110 using the slider 108 pulls the end of the fluidics line out of the lens as it is retained against the outlet of the insertion tube. Furthermore, the insertion tube 110 may have a coating to prevent any damage in case of contacting the lens.

After deployment of the lens into the eye, the fluidics system 102 is used to fill the lens to the specified volume by actuating one or more fluids, gases, gels, or solutes from one or more reservoirs 124. If the fluidics system 102 is located remotely from the handpiece 114 a fluidics line 104 may be used to move the fluid from the fluidics system 102 to the IOL 112. Refer to FIG. 1B for the system block diagram of the IOL insertion and filling system. The fluidics system may include one or more feedback systems 122 used to monitor pressure with a pressure sensor 126, flow with a flow sensor 128, or refractive index with a refractometer 130 and can adjust one or more variables through actuation of the pump to provide the appropriate refractive outcome of the lens. The pump actuation and feedback information is processed through a microcontroller 140 and appropriate software.

Deployment of the IOL occurs with the help of a viscoelastic in certain embodiments of the invention. The viscoelastic serves to reduce friction or stiction between the lens and the insertion tube. Likewise, in certain embodiments of the invention the viscoelastic is used as a carrier material that is pushed into the lens capsule by the injector and carries the lens along with it. In this manner, it supports the intraocular lens and assists the IOL to deploy into the lens capsule with the supported distal portions of the IOL entering first. The support of the viscoelastic prevents the flexible lens shell from buckling back on itself during insertion.

The viscoelastic assists in maintaining the lens capsule before insertion of the IOL. The viscoelastic is inserted into the lens capsule before or during IOL insertion and inflates the lens capsule to provide room for an inflatable IOL to be inflated. It displaces air from the injector and reduces or eliminates air bubbles from entering the eye that may be trapped in the folds of a deflated lens. The insertion tube 110 may be configured to be clear or translucent in order for the surgeon to visually inspect the lens during loading, while it is loaded, or during insertion. In FIG. 1 a slider 108 is used to deploy the IOL 112. However, this is not meant to be limiting and other approaches known to those skilled in the art can be used including other manual insertion devices such as a lever, ball screw, switch or an automated deployment through means such as pneumatic, motor, or solenoid actuation. After deployment of the lens into the eye, the fluidics system 102 is used to fill the lens to the specified volume. If the fluidics system 102 is located remotely from the handpiece 114 a fluidics line 104 may be used to move the fluid from the fluidics system 102 to the IOL 112.

Exemplary fluidics systems include a simple manual syringe or a fluidics pump, such as a syringe pump. The fluidics system 102 need not be an open-loop system; in certain implementations, feedback from a sensor is used to determine the fill volume, refractive properties of the lens as implanted in the eye, or pressure to fill to the correct volume. Fluidics system 102 may have the capability of both infusing fluid and aspirating fluid from the lens to reach the desired fill, refractive property, or lens pressure. In addition, fluidics system 102 may have the ability to monitor refractive properties of the lens filling fluid and adjust this.

Although the fluidics system is described as being remote from the handpiece, this is not essential. In certain implementations of the invention, the fluidics system is an integral part of the handpiece, any fluidic connections occurring within the handpiece. Other implementations that are within the spirit of the invention are possible to those skilled in the art.

Although insertion of the lens is described as the lens being pushed out of the insertion tube, it is also possible to retract the insertion tube 110 and fluidics line 104 and leave the lens 112 stationary. This has the distinct advantage of allowing the surgeon to place the IOL in the desired location, then retract the tube, exposing the IOL. Typically, in such embodiments, the fluidics line is 104 mechanically retracted before or along with the insertion tube. A blunt surgical tool, or another feature on the tip, may be used to hold the lens in place.

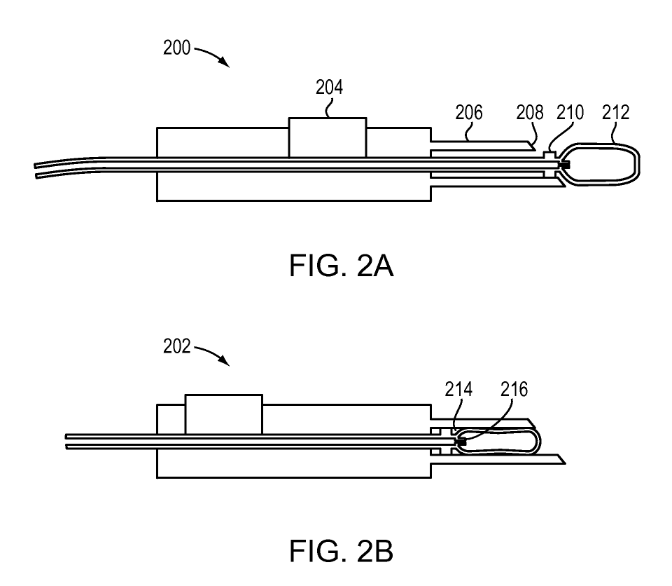

FIG. 2A illustrates an implementation with the IOL 212 deployed and FIG. 2B has the IOL 212 in the loaded configuration. In this implementation, a sealing plunger 210 forms a seal with the insertion tube 206. During loading a viscoelastic or other fluid, such as saline, balanced salt solution, or water may be used to assist in loading the lens. After the lens is loaded the intraluminal space 214 (which is bounded by the sealing plunger 210, the insertion tube 206, the IOL 212, and the end of the insertion tube 208) is filled with the fluid or viscoelastic. This filling fluid or viscoelastic is pushed out of the insertion tube 206 by the sealing plunger 210 along with the IOL 212 and fluidically pushes the lens from the insertion tube into the eye. In particular, forcing the fluid against the proximal side of the seal advances the plunger and pushes the lens out a known distance (until the seal has cleared the end of the insertion tube); again, a blunt surgical tool may be used to hold the lens and eject it from the fluidic line tip.

The filling fluid provides a fluidic force to assist in deployment the IOL 212 along with the mechanical force of the sealing plunger 210 along the proximal surface of the IOL 212. This is especially important for pushing out the unsupported distal end of the IOL 212 during lens deployment because it counteracts the tendency of the lens to become bunched up. The fluidic force also prevents the internal surfaces of the IOL 212 from being pushed against the access tip 216, which may cause damage to and possibly rupture of the IOL wall during deployment. The access tip 216 may be used to provide fluidic connection between the IOL 212 and fluidics system. The filling fluid reduces friction between the IOL 212 and the insertion tube 206 during deployment, thereby preventing damage to the IOL 212 during insertion. In addition, the filling fluid displaces residual air surrounding the IOL 212 and prevents the air from being pushed into the eye with the IOL 212. Air inserted into the eye with the IOL may rise to the top of the eye, stick to the lens, or enter the lens capsule making visualization of the insertion process difficult. The sealing plunger 210 also prevents damage to the IOL by stopping the proximal end of the IOL 212 from folding back and becoming pinched between the plunger and the internal surface of the insertion tube 206.

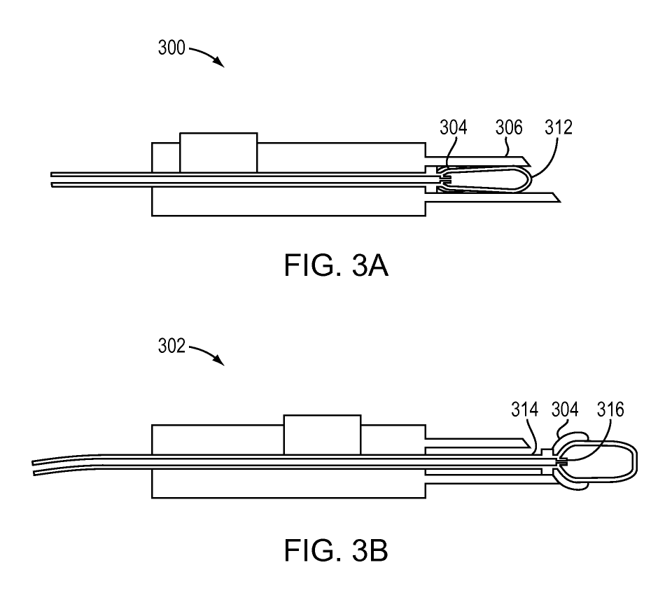

FIGS. 3A and 3B depict an implementation with a protective sheath 304 to assist in deploying the IOL 312. The protective sheath 304 wraps around a portion or the entirety of the IOL, and extends along a portion of the length of the IOL. In certain implementations, the sheath extends and covers the IOL lengthwise and circumferentially. In FIG. 3A the insertion tool 300 is in the loaded configuration and prepared for deployment into the eye. FIG. 3B shows the insertion tool 302 after insertion of the IOL 212, but before inflation of the IOL. The protective sheath 304 serves to protect the IOL 312 against frictional forces from the insertion tube 306. This is especially useful when the IOL 312 is made from a material that adheres to the insertion tube 306 or other surrounding structures. During deployment or loading of the IOL 312, the sides of the IOL may stick to surrounding structures, causing damage to the IOL 312. The protective sheath 304 serves as a carrier, and sliding friction occurs between the protective sheath 304 and the insertion tube 306. In addition, during loading of the IOL 312, the protective sheath 304 serves to pre-fold and/or roll up the lens while it is drawn into the insertion tube 306.

The protective sheath 304 may span the full length of the IOL 312, or a partial length of the IOL 312. In certain implementations, the protective sheath 304 is short, extending around a valve in the IOL 312. The sheath is used to hold the IOL 312 by the valve while the IOL 312 is drawn into the injector. This assists in drawing the lens into the injector and folding the lens. Deployment of the protective sheath protects the lens from damage by the access tip 316 by supporting the back portion of the lens, not allowing the front of the lens to fold over as it is deployed. In addition, the protective sheath 304 can be used to secure the valve before, during, or after insertion. Then, while mechanically retaining the valve, an access tip can be used to access the valve, providing fluidic continuity between the IOL 312 and the fluidics system.

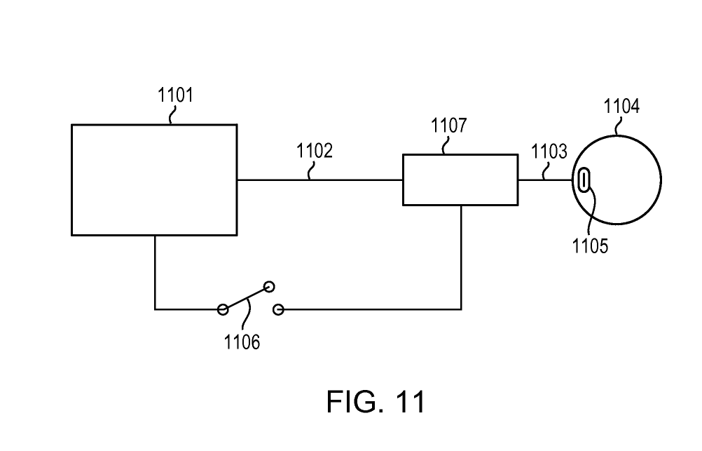

In certain implementations, the IOL 312 and protective sheath 304 are inserted together, then after insertion—but before, during, or after inflation of the IOL—the protective sheath 304 is retracted. In this manner, the protective sheath does not become trapped between the IOL 312 and the lens capsule after insertion and inflation. Likewise, the protective sheath may be used to load the lens into the insertion and filling system but is either partially deployed during lens insertion, or not deployed with the lens. In this implementation, the protective sheath 304 is used to fold and draw in the lens. To assist with this operation, the sheath may be shaped so as to promote folding of the lens (as described in greater detail in connection with FIG. 10). The material properties of the protective sheath 304 may be used to reduce friction between the IOL 312 and the insertion sheath 304 to allow smooth deployment. The protective sheath 304 then either does not come into direct contact with the lens capsule, or only slightly enters the lens capsule. In both cases this prevents damage to the lens capsule from the protective sheath 304.

Although the protective sheath 304 is described in connection with a liquid-filled IOL, this is not meant to be limiting. In certain implementations, this protective sheath is used with non-liquid-filled IOLs. When non-liquid-filled IOLs are used with the protective sheath, the fluidics system is not included in the design. Instead, a protective sheath is used in conjunction with an IOL injector to deploy the lens. This has the advantage of protecting the IOL during insertion from damage due to friction against the insertion tube, viscoelastic causing surface damage, or other damage from the compression experienced by the IOL during insertion. This type of sheath is especially important for micro incision IOL surgery, where IOLs are compressed to very small diameters, 2 mm or less, during insertion. Therefore, this concept of a protective sheath can be used to reduce damage for non-liquid-filled IOLs as well to ensure a safe deployment of the lens.

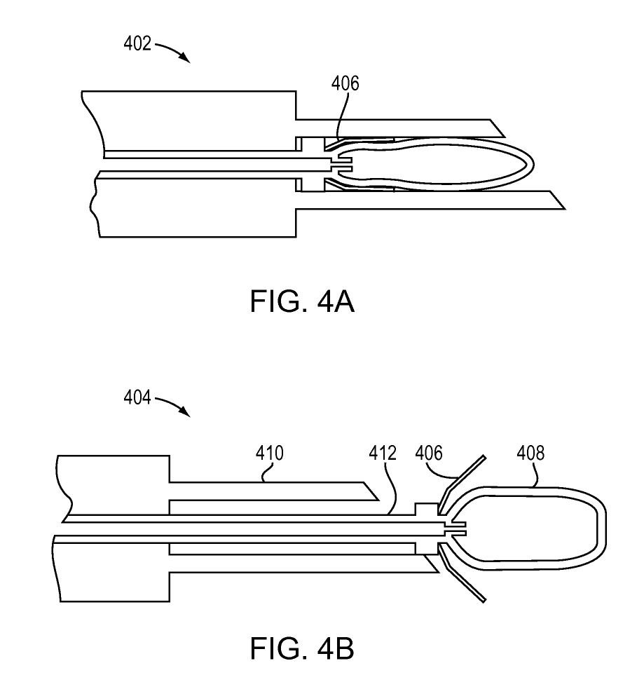

FIGS. 4A and 4B show an implementation with a mechanical gripping mechanism used to fold and deploy the lens. FIG. 4A has the IOL 408 in the loaded position while FIG. 4B has the IOL 408 in the deployed position. A mechanical gripping mechanism 406 is used to retain the IOL 408 on the insertion tube 412. This is useful, for example, if a valve is employed to communicate with the fluidics lines. The mechanical gripping mechanism 406 prevents the lens valve from becoming unconnected to the fluidics portion of the insertion and injection system.

In addition, the mechanical gripping mechanism 406 may be used to protect the lens during insertion. In certain implementations, the mechanical gripping mechanism 406 is configured similar to a forceps. In other implementations, the mechanical gripping mechanism 406 is soft or flexible, made of a polymer (such as a silicone) to engage the IOL 408 without causing damage thereto. In addition, a soft material is preferable to prevent damage to the lens capsule after insertion of the IOL into the eye. The flexible gripping mechanism 406 may comprise or consist of two or more elements to grasp the IOL 408. As shown in FIG. 4B, the mechanical gripping mechanism 406 allows release of the IOL 408 after insertion. If the mechanical gripping mechanism 406 is configured like a forceps, upon deploying the lens, the gripping mechanism 406 automatically opens. For example, the grippers may be spring-loaded or include living hinges biased toward an open, spread-apart configuration, so that when they are deployed, they spread out. The gripping mechanism is structurally limited to only open a set distance which is large enough to release the lens, but smaller than the incision (less than 3 mm, and in some cases less than 1 mm). The mechanical gripping mechanism may be retracted after delivery of the IOL 408, before, during, or after filling the IOL 408.

In addition, a gripping mechanism may be used for accessing a deflated, partially inflated, or completely inflated IOL after insertion into the eye. When used in this manner, the gripping mechanism may be biased in the opposite direction or be configured to to draw the grippers toward each other; see, e.g., U.S. Ser. No. 61/920,615 (filed on Dec. 24, 2013), the entire disclosure of which is hereby incorporated by reference. The trippers may mechanically hold the lens while a valve in the IOL is accessed. At this point fluid can be added or removed from the IOL. This provides the possibility of implanting an unfilled IOL, then after implantation accessing the valve and inflating the lens. In this situation, the IOL is not in fluidic connection with the filling lines during implantation.

Other suitable gripping mechanisms access a valve in a fluid-filled IOL. One exemplary mechanism utilizes vacuum to retain the valve or by mechanical holding pressure; for example, the mechanism may utilize a pair of concentric tubes, the inner one extending beyond the outer one and being insertable into the lens, with the vacuum being applied through the outer lumen to draw the lens against the distal end of the outer tube. The valve may be accessed directly with a small tube or needle. Some implementations of the invention mechanically retain the valve and then use a fluidic pressure to crack the valve open to either add or remove fluid from the liquid-filled IOL.

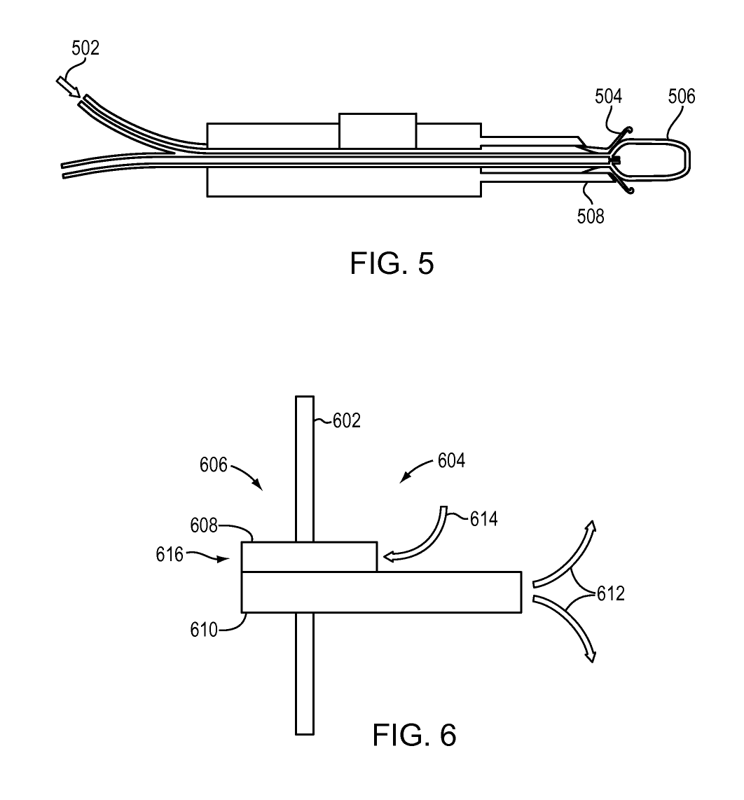

FIG. 5 shows an implementation with a fluidic line used to fluidically push the IOL 506 out of the injector. Fluid from an inlet 502 enters the insertion and filling system and exits through the insertion tube 508. During insertion of the IOL 506, the fluid flows the IOL out of the insertion tube 508 without forcing the IOL to fold onto itself. In addition, the fluid can be used to inflate the lens capsule. This fluid can be used instead of or in support of viscoelastic that is on or around the lens or inside the lens capsule. In certain implementations, the fluid displaces viscoelastic in the lens capsule after insertion of the IOL 506. This is especially important when an IOL is sized to fill most of the lens capsule. After inflation of a large lens-capsule-filling IOL, viscoelastic may become retained between the IOL wall and the lens capsule. Therefore, either avoiding use of viscoelastic or cleaning viscoelastic from the lens capsule during insertion and implantation may become appropriate.

Although FIG. 5 shows the additional fluidic line being coupled through the insertion tube, in other implementations the fluidic line is on the outside of the insertion tube and is used not as a source of fluidic force to push out the lens, but to inflate the lens capsule and/or clean out viscoelastic during insertion of the lens. In other implementations, an external aspiration line is used in conjunction with the external fluidic infusion line. Infusion and aspiration may be used together to remove any fluid, such as viscoelastic, from the eye. The infusion line may be coupled to the insertion tip, or may be external to the insertion tip. Likewise, the infusion and aspiration may be separated from the insertion tip, e.g., in the form of separate handpieces working together to exchange fluids in the eye.

Refer now to FIG. 6, which depicts an access tip in the form of a dual cannula providing both infusion and aspiration. The access tip 616 is placed from outside the lens 606 into the inside of the lens 604. An infusion portion of the injection tip 610 is used to infuse fluid 612 into the lens. A second port is used for aspiration 608 to aspirate the contents of the lens 614. This aspiration port 608 need not be located directly adjacent to the injection port 610. In certain implementations of the invention the access port and infusion port are located on opposing sides of the lens, and are put into the lens through two distinct access points. When infusion and aspiration are used together, it is possible to exchange fluid in the IOL. This is useful, for example, when changing the refractive index of the fluid filling the IOL. Likewise, feedback systems in the handpiece can be used to monitor pressure, flow, or refractive index and the handpiece can adjust a single one or a combination of these to provide the appropriate refractive outcome of the lens.

Some implementations of the access tip utilize a blunt tip with multiple lumens configured in concentric or parallel orientations for infusing or aspirating fluid from the side of the tip. Still other implementations of the access tip involve features to prevent the IOL from collapsing over the aspiration hole. Exemplary access tip features include side ports, multiple lumens, and a rounded tip. This may be important, for example, when the IOL is evacuated prior to insertion into the eye. In this situation, a flexible wall of a liquid-filled IOL may cause lumen occlusion. However, a feature such as a protruding member or multiple lumens can be used to prevent lumen occlusion.

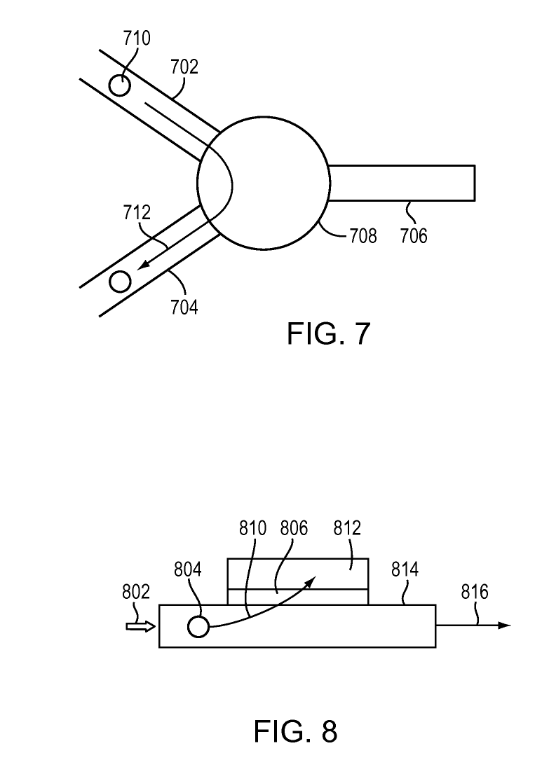

FIG. 7 depicts a separate infusion line 702 and aspiration line 704 attached to the access tip 706 through a y-connector or valve 708. An air bubble 710 travels through path 712 from the infusion line 710 and passes through the y-connector or valve 708, then passes out the aspiration line 704. Fluid traveling along this path does not enter the access tip 706. In this manner, the lines of the insertion and filling system can be primed up to the injection tip 706 without passing fluid into the injection tip 706. For example, the valve 708 may selectively connect the line 702 to the line 704 or line 706, so that air is cleared from the line 702 (via line 704) before it is connected to line 706. In some embodiments, the valve 704 is positioned higher than the line 706 so that the air travels out as gases tend to accumulate on the top of the line. Although FIG. 7 is shown with air bubbles, this approach also applies to any air in the line that can be removed.

Refer now to FIG. 8, which depicts a debubbling filter used with the injection tip. Liquid from the fluid reservoir moves through the infusion line 814 in a direction depicted by arrow 802. Air bubble 804 flows down the infusion line 814 until coming in contact with semipermeable membrane 806, which allows air to cross but blocks liquid from crossing. Air bubble 804 traverses the semipermeable membrane 806 via path 810. Air enters a separate chamber or line 812 after removal from the line. In this manner, liquid traveling out of the distal end of the infusion line 816 and into the IOL is free of air bubbles. Semipermeable membrane 806 may also be used to remove air during priming. Chamber 812 may be at ambient pressure (if the liquid in the line 814 is at higher pressure), or held under vacuum. Likewise, the driving force for air to leave may be a pressure differential from the infusion line 814 and the chamber 812, or the process may be from diffusion.

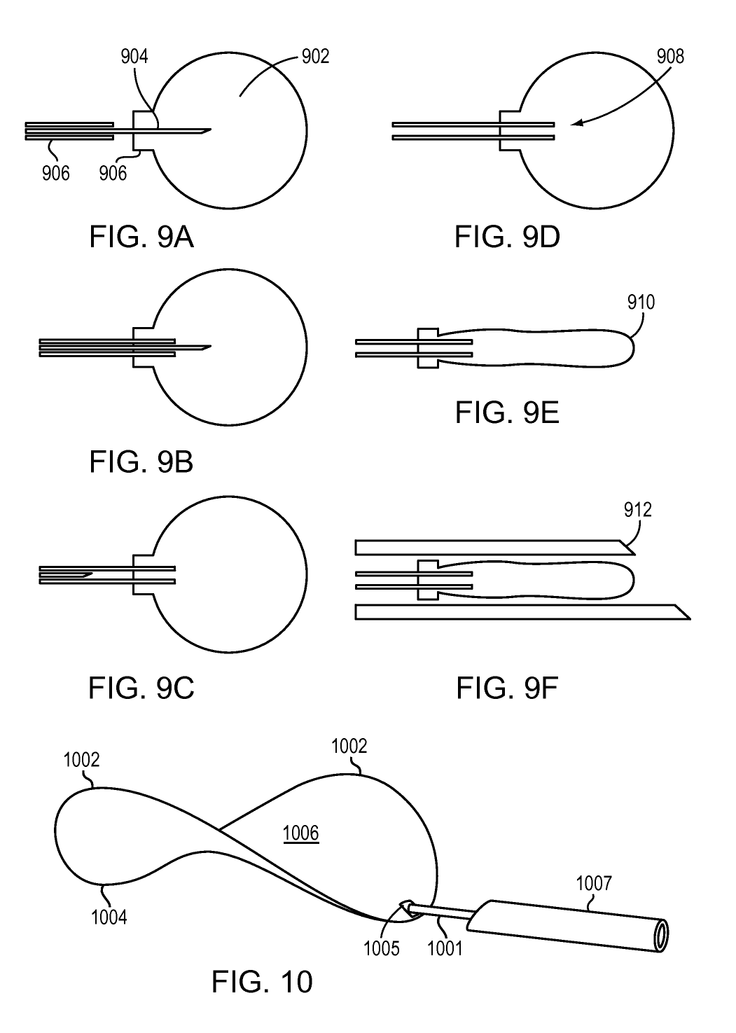

FIG. 9 illustrates an exemplary method of inserting an IOL 902 onto the injector. The lens is checked for leakage after insertion onto the injection and filling system. In FIG. 9A, a sharp needle is first used to access or pierce a sealing portion 914 on the IOL. Then, as shown in FIG. 9B, the access tip 906 is inserted through the sealing membrane 914 into the IOL. Fluidic continuity between the fluidic system and the inside of the IOL 902 is achieved at this step. In FIG. 9C, the sharp needle is removed from the IOL. In FIG. 9D, the IOL is inflated with air or liquid to assume an inflated state 908. At this point the inflated IOL 908 is checked for leaks or damage to the IOL. This detection may be performed, for example, by optically inspecting the lens for deflation; by visually inspecting the lens for leakage; by monitoring pressure of the lens; or by monitoring fluid flow to and or from the lens. These techniques are not meant to be limiting and many other similar techniques known to those skilled in the art may be used to inspect the lens. In FIG. 9E the IOL is deflated and is in the deflated state 910. In FIG. 9F the IOL is inserted into the insertion tube 912. FIGS. 9A-9F illustrate an exemplary approach for checking the lens for leaks, but the illustrated steps are not meant to be limiting. For example, the lens may be accessed without a sharp tool 904 to check for leakage. In addition, the lens may be checked for leakage and subsequently removed from the injection and filling system for later use.

Viscoelastic can be used to deploy the IOL. Viscoelastics are used to maintain space between the IOL and the surrounding injection tubes. In addition, they assist in sealing portion of the injector when inserting the lens. This is true when a close fit is between a portion of the injector and the injector wall. In certain embodiments of the invention, the viscoelastic plugs a plunger used to deploy the lens. As the viscoelastic moves, it draws the light lens shell with it into the eye. In addition, the viscoelastic lowers friction and reduces stiction between the lens and surrounding insertion tube. Finally, during insertion into the lens capsule, the viscoelastic may enter the lens capsule before or simultaneously as the IOL enters the lens capsule. In this case the viscoelastic maintains the lens capsule in the inflated position and provides a space for the lens to sit inside the lens capsule. This is important during filling of the lens so there is a space for the lens to easily fill out, reducing wrinkling of the lens or lens capsule during insertion.

Viscoelastics are also used to fold thin walled injectable lenses. By placing a thin line of viscoelastic along a diameter of the lens corresponding to the fluidic line, the lens can be folded around this line enclosing the viscoelastic. The viscoelastic in this embodiment of the invention acts as a guide to roll up the thin walled IOL for retraction into the injector and injection into the eye. This prevents unwanted IOL folding during retraction into the injector and injection into the eye.

Suitable viscoelastics include, but are not limited to dispersive and cohesive viscoelastics or a combination of these. Exemplary viscoelastics include include hydroxypropyl methylcellulose solutions such as OcuCoat, sodium hyularonate solutions such as Provisc, chondroitin sulphate I sodium hyuronate soultions such as Viscoat. Other exemplary viscoelastics include HEALON, HEALON 5, HEALON GV, HEALON EndoCoat, Amvisc, Amvisc Plus, Medilon, Cellugel, BVI 1%, StaarVisc II, BioLon, and ltrax. Examples of combinations of viscoelastics include mixtures of dispersive and cohesive viscoelastics (e.g. DuoVise which contains separate syringes of Viscoat and Provisc) or HEALON Duet Dual (consisting of HEALON and HEALON EndoCoat). As an example, a dispersive viscoelastic may be used to cover the lens, while a cohesive viscoelastic is used around the dispersive to carry the IOL into the lens capsule. The IOL can be loaded into the injector in a number of ways known to those skilled in the art, including, but not limited to, front and back loading and closing the inserter around the IOL. Once loaded, the injector may be stored under standard IOL storage conditions until use.

In various loading embodiments, the lens is loaded using unique features of the IOL and the peripheral system. FIG. 10 depicts a fully evacuated fluid-filled intraocular lens. The access tip 1001 is used as a fluidic connection between the fluid-filled intraocular lens 1012 and the filling system. The access tip 1001 connects to the fluid-filled intraocular lens 1012 through a valve 1005 that creates a sealed fluidic connection thereto. The fluid-filled intraocular lens 1012 naturally conforms to a saddle shape, since that is theoretically the lowest surface-energy configuration due to its geometry. The access tip 1001 can protrude into the lens and flatten the curve though the center of the saddle slightly depending on how far the access tip extends. During loading, the edges 1002 and 1003 are folded over towards the center of the lens. This makes the lens form what is similar to a rolled tubular shape or a “taquito.” There are ways to help the fluid-filled intraocular lens fold into this loaded position. One technique is to lay a fluid (preferably a highly viscous liquid such as a viscoelastic) across the center channel of the lens, which starts at the end of the lens 1004 and extends through the center channel 1006 and up to the valve 1005. This allows the edges of the lens 1002 and 1003 to fold over a medium to prevent excess stresses to specific regions of the IOL during folding. Additionally, the surface tension of viscous fluid promotes the edges to fold over. The second technique uses the insertion tube 1007 in which the lens 1012 is loaded into to help it fold over itself during the loading process. The angled taper on the insertion tube 1007 helps first feed the valve portion 1005 of the fluid-filled intraocular lens first. As the lens is pulled farther and farther back into the insertion tube 1007, the tapered side walls of the tube opening slowly push the sides of the lens 1002 and 1003 over each other. This can also be achieved by placing a funnel in front of the insertion tube 1007 that will hold the lens. The funnel can then be detached after the lens is fully loaded into the insertion tube 1007. A third technique to help the lens load is to use a sheath that can wrap over the valve 1005 portion of the fluid-filled intraocular lens 1012. As the lens 1012 is pulled back into the insertion tube 1007, the sheath slowly curls over the lens and helps the lens fold over. The sheath also protects the fluidic connection by wrapping itself around the valve 1005 area of the lens. The sheath prevents the insertion tube 1007 from applying friction to the valve area. Such friction may prevent the valve from being loaded smoothly into the insertion tube 1007, subsequently causing the fluidic connection to be disconnected during loading or damage to the lens 1002.

A second embodiment back loads the intraocular lens 1012 through the insertion tube 1007. With this approach, the lens is pushed from the back of the tube to the front where it is ready to be injected. A funnel can be used to help guide the lens into the insertion tube 1007 in this approach as well. If the lens is back-loaded, a surgical tool with a grabbing mechanism such as forceps can be placed through the insertion tip from the front where the angled cut is. The grabbing mechanism can then go through the insertion tube tip and grab onto the end of the lens 1004. The lens can then be pulled through the insertion tube 1007 to be back loaded. This is to help the lens fold correctly and to prevent the lens from inappropriately folding within the insertion tube 1007. The end of the lens 1004 may have an additional segment to be preferably grabbed by the forceps. The forceps may be coated with a polymer such as silicone to prevent any damage to the lens 1012 during contact.

Either approach may be used to load a cartridge for storage. The cartridge may then be placed within an accessible portion of the insertion tube prior to implantation. The access tip 1001 is connected to the IOL 1002 to create a fluidic connection prior to the procedure.

### 2. Re-Access

FIG. 11 illustrates a fluid-filled intraocular lens 1104 already implanted in a patient's capsular bag or in the cliliary sulcus. One or more access ports 1105 are located on the surface of the fluid-filled intraocular lens 1104, preferably outside of the field of vision. The access port 1105 allows an access tip 1103 to enter or pierce though and access the fluid within the fluid-filled intraocular lens 1104. In one embodiment, the access tip 1103 has an overall diameter less than 4 mm, and ideally less than 2 mm in order for the access port 1105 to maintain its self-sealing properties and to minimize leakage during or after access. This access tip 1103 can be manipulated using a handpiece 1107, allowing the surgeon to operate mechanisms to control the access tip 1103 orientation, length, and fluid transfer rate. One or more fluidic lines 1102 connect to the access tip 1103, and runs through the handpiece 1107. The fluidics line 1102, then connects to a console 1101. The console 1101 uses a pumping mechanism (e.g., a mechanical pump, syringe pump, peristaltic pump, or other pumping mechanism that is preferably meterable) to add fluid, remove fluid, or add and remove fluid sequentially or simultaneously. The surgeon can control the different injections and removal of fluid by a switch 1106, which can either be a foot pedal or pedals, hand controls, or some combination of both. To maintain convenient control of the handpiece, the line may be flexible, thereby allowing the surgeon to move the handpiece easily while accessing the intraocular lens. Due to the sensitivity and accuracy of fill that may be required of a fluid-filled intraocular lens 1104, the fluidics line 1102 may have minimal wall compliance and be designed for pressures above 10 psi. The fluidics line 1102 will endure high pressures (above 10 psi) during injection as most of the pressure drop occurs across the access tip 1103. The Hagen-Poiseulille equation,

$$Δ P = \frac{8 μ LQ}{π {r}^{2}}$$

(where ΔP is the pressure drop across the tube or pipe; μ is the dynamic viscosity; L is the length of the tube; Q is the volumetric flow rate; and r is the inner radius of the tube), shows that the majority of the pressure drop occurs through the access tip since the access tip has a much smaller inner diameter than the fluidic line. This means the fluidics line is under higher pressure while fluid is flowing through the line. More specifically, the line compliance may be designed for pressures between 10 psi and 1000 psi. These internal pressures expand the inner diameter of the fluidics line, and this expansion creates the compliance in the line by changing its volume. These compliances can be estimated by using basic equations of thin-walled pressure vessels. In some cases, thick-walled open-ended pressure vessel equations may be used. Fluidic line compliance may be important in re-access operations that modify internal liquid quantities of 2 μL or less. For example, if the fluidics line 1102 expands from an inner diameter of 0.010″ to 0.011″ and is 3′ in length, the compliance in the system would be about 39 μL. Nominal total fill levels of the intraocular lens are between 10 μL and 700 μL, and preferably between 50 μL and 250 μL. This means the volumetric change within the fluid line is 39 μL from when the system is relaxed to pressurized. In this instance the surgeon must wait a designated amount of time after the injection has been made to account for fluid line compliance and/or monitor fluid flow or lens properties, such as refractive state, internal pressure, or refractive index of the fluid directly at the lens or proximal to the tip. This effect of waiting for the line to relax can be seen in the physiology compliance equations ΔP×C=ΔV, where ΔP corresponds to the change in pressure, C is the compliance and ΔV is the change in volume. Waiting for the line to relax allows the fluid to reach equilibrium and stop flowing, making ΔP=0. Therefore the compliance has no effect of the volume change. In another approach, the wall of the fluidics line 1102 may have a negligible compliance. This means the walls of the line are stiff enough that they do not expand under pressure. The fluidics line 1102 would still have to maintain its flexibility to allow the surgeon to manipulate the access tip 1103.

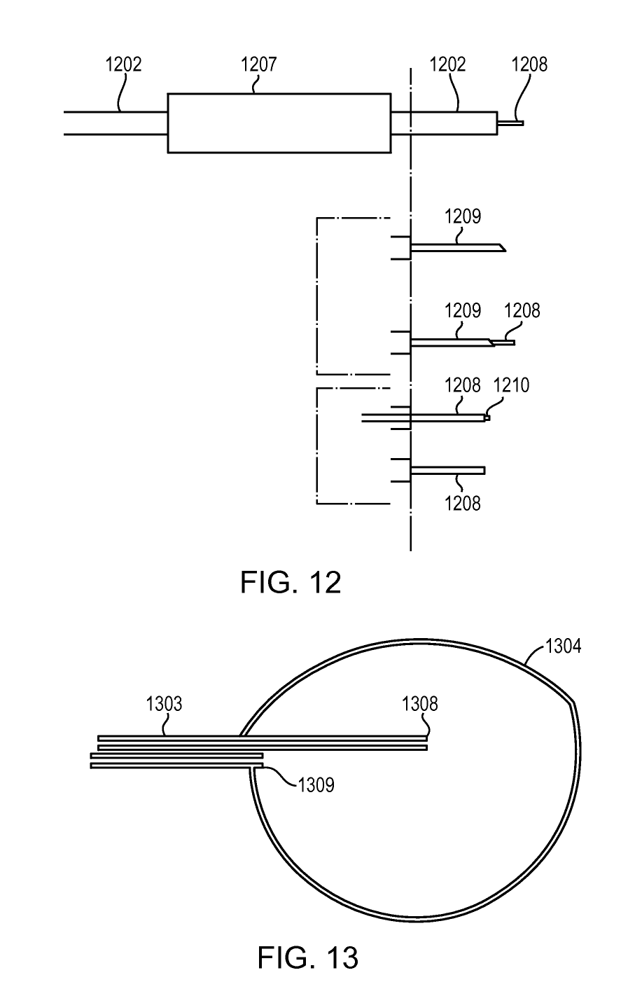

In the configuration shown in FIG. 12, the fluidic line 1202 still runs through the handpiece 1207, but the figure illustrates some of the different configurations that an access tip can take. In the top form, the fluidics line 1202 connects directly to a smaller tube, which is the access tip 1208 that would either pierce through the valve or enter a passage. In this configuration, a corneal incision is made into the eye to allow the access tip 1208 and fluidics line 1202 to access the fluid-filled intraocular lens. The fluidics line 1202 may be less than 4 mm in overall diameter so that the surgeon can either re-open the initial incision used to insert the fluid-filled intraocular lens or make a new incision small enough to avoid inducing astigmatism. The access tip 1208 may either have a locating device to position the access tip to go through an access port or may have a sharp point, permitting it to break through a valve membrane to access the fluid-filled intraocular lens. With reference to portion A in FIG. 12, the access tip 1208 is incased and protected by an outer tube 1209. This tube has a sharp point at its end. This allows the surgeon to pierce the eye, e.g. through the cornea, and move the outer tube 1209 into position to access the fluid-filled intraocular lens. The access tip 1208 is then deployed from the outer tube 1209 and accesses the fluid-filled intraocular lens. In this configuration, the sharper outer tip 1209 does not contact the intraocular lens, but is used to create an incision in the eye. In the configurations associated with portion A of FIG. 12, the surgeon does not have to make a corneal incision. In the configurations associated with portion B of FIG. 12, a sharp point 1210 protrudes out of the access tip 1208 and helps cut through the eye to the fluid-filled intraocular lens. This configuration also does not need a corneal incision. The point may cut through statically (i.e., the surgeon pushes the point through the eye) or may cut dynamically. In the latter case, the sharp point 1210 may be excited by ultrasonic energy or reciprocate relative to the access tip 1208 to cut through the eye. In both configurations the sharp point may or may not also help access the fluid-filled intraocular lens through and access port or membrane.

Once the fluid-filled intraocular lens is accessed, the sharp point may be withdrawn and fluid removed, added, or exchanged. In certain embodiments, the sharp point 1210 is put in a first position in which it extends beyond the access tip 1208 upon entering the valve of the intraocular lens. Then, prior to accessing the intraocular lens valve, the sharp point 1210 is retracted to a second position inside the fluidics line 1202, thereby preventing flow obstruction in the access tip 1208 during infusion or aspiration of fluid. In other embodiments of the invention, the sharp point 1210 is used to keep the access tip 1208 rigid during insertion into the valve.

FIG. 13 illustrates a dual-lumen access tip 1303. In this configuration, the first lumen 1308 is further inserted within the IOL relative to the second lumen 1309, thereby facilitating proper fluid mixing when the internal contents of the IOL 1304 are exchanged by simultaneous or sequential infusion and extraction of fluid.

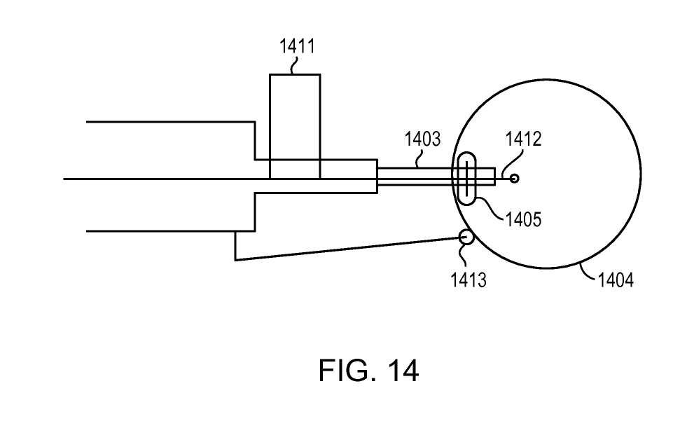

FIG. 14 illustrates a feedback configuration that allows a microprocessor to measure the amount of fluid that needs to removed, exchanged, or injected from fluid-filled intraocular lens 1404 through an access port 1405. A flow sensor 1411 or other metering device is placed near the access tip 1403. The position of the flow sensor is critical due to the compliance that may be in the fluidics line as explained previously. Alternatively, if fluid is being removed through a vacuum, then due to cavitation and compliance of the lines the sensor 1411 should be placed as close to the access tip 1403 as possible. All of the fluid volume in the access tip 1403 and fluidics line represents dead volume. This dead volume may also be used a measurement. If a known amount of fluid needs to be removed, the access tip 1403 may be designed to accommodate exactly that much liquid; as soon as the liquid reaches the sensor 1411, the removal of fluid is complete.

Another useful feedback parameter is the pressure of the fluid-filled intraocular lens 1404. This may be measured by feeding a small pressure sensor through the access tip 1403 and into the fluid-filled intraocular lens 1404. A fiber-optic pressure sensor may be used for this purpose, for example. Another configuration is a probe 1413 that extends either from the fluidics line or the access tip and pushes against the wall of the fluid-filled intraocular lens 1404. The force, deflection, or both can be measured and fed back to a processor to help control the injection, exchange, or removal of fluid. In other embodiments, tonometry—such as applanation tonometry, Goldmann tomonetry, dynamic contour tonometry, indentation tonometry, rebound tonometry, pneumatonometry, impression tonometry, or non-contact tonometry using an optical device such as optical coherence tomography—may be used.

Another configuration not shown in FIG. 14 measures in real-time the power of the fluid-filled intraocular lens 1404 using wavefront aberrometry, refractometry, autorefractometry, ultrasound measurement of lens dimensions, and/or optical coherence tomography of lens dimensions. This parameter is fed back to a processor to help control the injection, exchange, or removal of fluid. For example, lens geometry may be used with a measured refractive index of the fluid. The refractive index may be adjusted to produce emmetropia of the patient. In another embodiment, the fluid amount is used with measurements of anterior and posterior lens curvature, position of the lens relative to the retina and cornea, a prior measurement of corneal power, and the fluid level, or refractive index is adjusted to produce emmetropia. In other embodiments of the invention, the pressure of the intraocular lens is monitored to ensure a conformal fit between the surrounding lens capsule, and the refractive index of the intraocular lens is monitored to adjust for emmetropia.

Not pictured in the figures is a locking or locating mechanism to secure the re-access connection during fluid exchange. This mechanism allows the access tip to pierce through and into the liquid filled intraocular lens and maintain such configuration. Suitable locking mechanisms include but are not limited to snap locks, twist locks and slide locks. Suitable locating mechanisms include but are not limited to tethers, vacuum (onto a surface having a unique shape), grippers or pins with locating holes. One configuration utilizes an existing self-sealing hole; the access tip uses the locking and/or locating mechanism to align with the hole, and is then be pushed through the hole to access the liquid inside the lens. In another configuration, the access tip pierces straight through a membrane or valve into the lens. In certain embodiments of the invention, a locking mechanism is used to prevent a pushing force during the valve access procedure from causing the lens to move and strain surrounding tissue. First the tool is locked to the locking mechanism, which allows the lens to be held in the appropriate position without straining surrounding tissue. Next the access tip is used to access the valve.

### 3. Removal

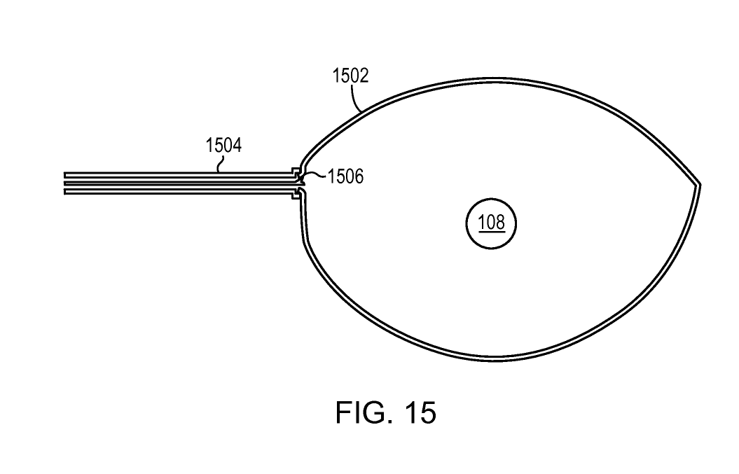

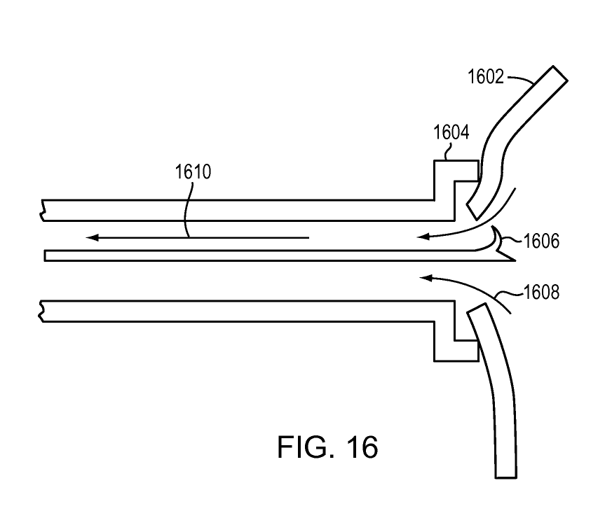

Refer now to FIG. 15, which depicts an exemplary IOL explantation system 1504. The explantation system 1504 grabs onto and retains the side of the liquid-filled IOL 1502. Upon retention, an internal tip is used to access the inside of the IOL and aspirate the fluid 1508 from the IOL into the explantation aspiration tool through a fluid path 1506. FIG. 16 shows a close view of the explantation system. In the illustrated implementation, a mechanical gripper 1604 is used to hold onto the IOL lens wall 1602. The IOL lens wall 1602 may be a specific portion of the IOL meant to interact with the gripper. In certain implementations this portion of the IOL contains a locking mechanism that interacts with the gripper. In other implementations, the gripper interacts with a valve in the lens. Upon mechanically contacting the lens and retaining it, either through mechanical force or by suction, the lens-access portion 1606 of the explantation system is used to access the lens. This causes the silicone oil or other liquid inside the IOL to flow from the lens into the explantation tool along fluid path 1608. The explantation tool applies aspiration to remove the internal contents of the lens. The gripping and aspirating system allows the internal contents of the lens to be aspirated without coming into contact with other ocular structures.

In certain embodiments, the access portion 1606 is a barbed hook, sharp point, crescent hook, or forceps and is used to access the internal contents of the lens. In other embodiments, the lens-access portion 1606 is a cannulated structure such as a cannulated hook or needle. Aspiration of the IOL contents occurs through the cannulated structure and/or through the surrounding explantation tool. In other embodiments, the access portion 1606 comprises a hollow structure that aspirates through a series of ports. When the flexible lens collapses on the access portion 1606, the other ports continue to aspirate. In one embodiment, features on the access portion, such as one or more small protrusions, prevent the deflated lens from closing off the apertures in the access portion 1606. The access portion 1606 of the device is not meant to be limited by descriptions above; it can be any cannulated on non-cannulated instrument that is used either to open an aperture in the lens or to sample the lens contents.

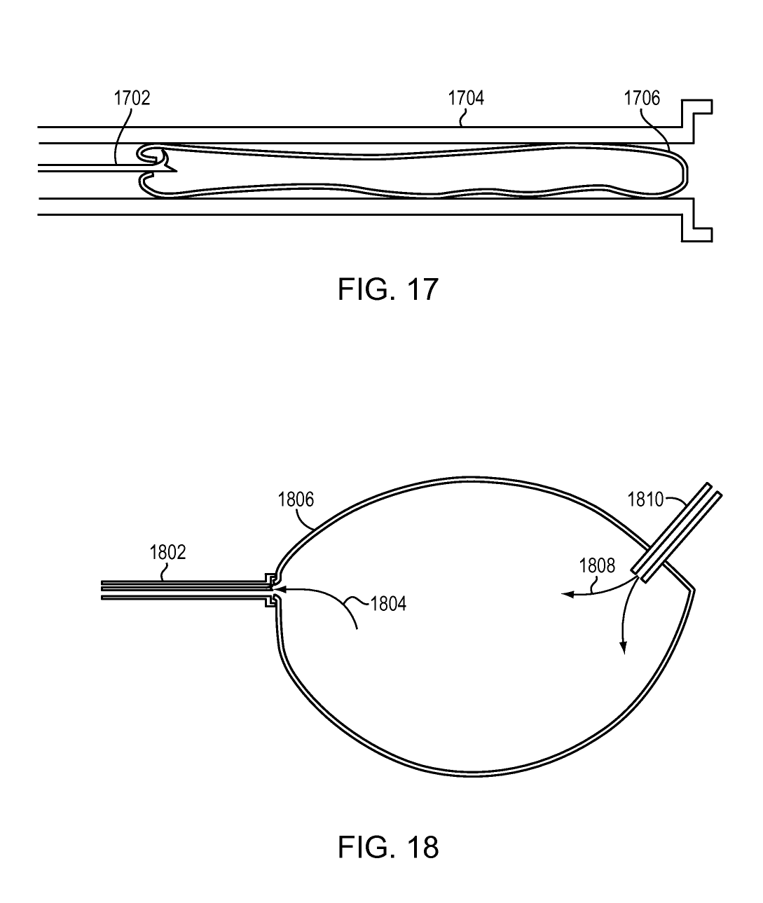

Refer now to FIG. 17. After aspirating all of the contents of the IOL, the IOL 1706 is brought into the explantation system 1704 in a deflated state. In certain embodiments, a mechanical retaining device, such as a hook or barb 1702, is used with or without aspiration to assist in drawing the deflated IOL 1706 into the explantation system 1704. In other implementations, a dual-lumen or coaxial access portion of the explantation tool is used to access the lens. One portion of the dual-lumen/coaxial tool infuses a liquid while the other removes the fluid inside the lens through aspiration. This allows the filling liquid to be replaced with another liquid, such as a lower-viscosity liquid, or a liquid that is better tolerated in the eye (such as a balanced saline solution or viscoelastic) before the lens is deflated. In this manner, the lens remains partially or totally inflated during removal of the internal contents of the lens. Then, after fluid exchange has occurred, the internal contents are aspirated out and the lens is removed.

FIG. 18 shows an embodiment of the invention with a bimanual explantation tool. Aspiration and removal of fluid from the lens is performed with the aspiration portion of the explantation tool 1802. This portion of the tool may be configured as described above. Fluid from inside the IOL travels along fluid path 1804 into the aspiration portion of the explantation tool. An infusion portion of the explantation tool 1810 is used to access another portion of the IOL 1806. While the lens contents are aspirated using the aspiration portion of the explantation tool 1802, the IOL 1806 volume is filled with fluid flowing along path 1808 from the aspiration portion. During this procedure, the contents of the IOL are exchanged with another fluid or fluids. Exemplary fluids include balanced salt solution, viscoelastic, or air. After fluid exchange has occurred, the lens is emptied and brought out of the eye using either the explantation tool itself or a secondary tool such as a forceps.

In some embodiments the lens is partially deflated while a second tool is used to fill the lens capsule with viscoelastic to maintain the size of the lens capsule. In this manner, the lens capsule size is retained while the IOL is deflated. This procedure protects the lens capsule from damage while the IOL is removed and allows a second IOL to be implanted into the already full lens capsule. The large size of a fluid-filled IOL helps to maintain an open lens capsule, making lens exchange into the lens capsule an easier and safer procedure than with smaller-profile IOLs.

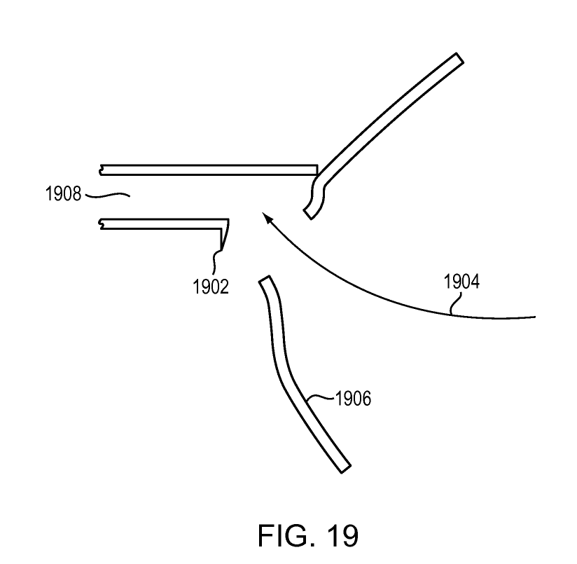

FIG. 19 illustrates an explantation tool with a sharp portion 1902 that is used to open an aperture in the IOL 1906. Aspiration from the lumen 1908 of the explantation tool is used to remove any fluid from the IOL. Fluid from the inside the IOL passes along a fluid path 1904 from the IOL to the explantation tool. In one embodiment, the explantation tool provides infusion and aspiration. Infusion maintains the intraocular pressure and stabilizes the anterior chamber while aspiration removes fluid from the IOL. In other embodiments, a sharpened tool, which is a separate part of the explantation system is used to open an aperture in the IOL while an aspiration or infusion-and-aspiration portion of the explantation tool is used to aspirate the contents of the IOL. Then the empty IOL is removed using a separate tool or through the aspiration portion of the explanation tool. In certain embodiments, the IOL is filled with a fluid less dense than the surrounding aqueous. This is advantageous because such fluid tends to rise to the top of the eye, easing removal of fluid. In addition, if the lens capsule is damaged during the explantation, the lens floats to the top of the eye, preventing fragments from entering the vitreous chamber.

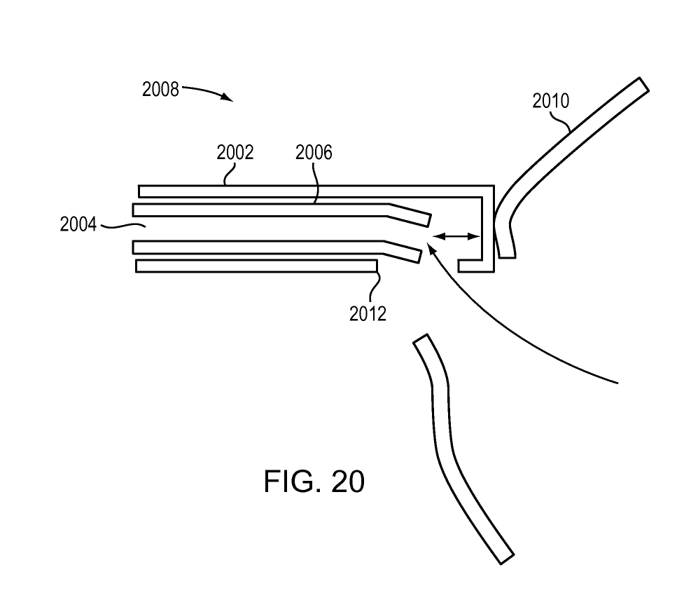

Refer now to FIG. 20, which shows an explantation system 2008 comprising a cutting tool used to cut a portion of the lens and aspirate the lens and filling fluid. The explantation system 2008 has an outer tube 2002 with a cutting port 2012 and a cutting blade 2006 located telescopically within the outer tube 2002. In a configuration shown in FIG. 20, the cutting blade 2006 reciprocates linearly inside the outer tuber 2002. However, reciprocating linear motion, reciprocating rotary motion, rotary motion, or a combination of two or more of these motions are all within the scope of the invention. The lens 2010 is opened b y the cutting motion of the explantation system 2008. Then the liquid contents of the explantation system are aspirated out of the eye through the lumen 2004 of the cutting blade 2006. Suction is applied to the inner lumen 2004 of the cutting blade 2006 to draw in the lens and lens fluid. In certain implementations, the cutting blade 2006 contains a sharpened edge to assist in shearing a portion of the lens. In other implementations the cutting blade 2006 contains a bend or spring-loaded mechanism to create a shearing force between the cutting blade 2006 and the outer tube 2002.

Other techniques to open an aperture in the lens and aspirate out the lens fluid include using an ultrasonic probe along with a tube used as a cutting tip, and applying suction through the center of the tube. For example, an ultrasonic probe may be located coaxially and external to the cutting tip, which may include a feature for breaking the lens. In certain embodiments, the lens-breaking feature comprises or consists of a beveled edge, sharp point, angled point, or a sharp edge. Alternatively, a laser may be used to open an aperture in the IOL. The laser may be externally or endoscopically applied to the lens. Certain implementations of the invention include infusion and/or aspiration with the laser source to evacuate the contents of the lens before lens removal. Another approach uses cautery to open an aperture in the IOL and aspiration to remove the lens filling liquid. Likewise, certain implementations of the invention include infusion as well as aspiration. For the above-mentioned variations, it is possible to remove the lens with forceps or another manual tool, or with the extraction system and tool itself.

Certain embodiments of the present invention have described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

1. An intraocular lens insertion and filling system comprising:
a fluidic system including one or more pumps and one or more reservoirs for a liquid;
a conduit, fluidically connected to the pump and having a distal end configured for insertion into an intraocular lens;
an insertion mechanism including a handpiece terminating in an insertion tube, wherein:
the handpiece surrounds a distal portion of the conduit;
the insertion tube is configured to receive the lens in an at least partially deflated state; and
the handpiece includes an advancement mechanism for causing relative movement between the insertion tube and the lens received therewithin, whereby activation of the advancement mechanism causes the lens to be ejected from a distal end of the insertion tube.

2. The system of claim 1, wherein the pump is adapted to pump a liquid from the reservoir into the lens following ejection thereof from the insertion tube, thereby inflating the lens.

3. The system of claim 1, wherein the fluidic system comprises a pressure sensor for measuring an internal pressure of the intraocular lens during inflation thereof.

4. The system of claim 1, wherein the pump is a bidirectional pump, and further comprising a flow sensor for measuring an amount of liquid introduced into or withdrawn from the lens by the pump.

5. The system of claim 1, wherein the fluidic system further comprises an inline refractometer for measuring a refractive index of the fluid inside the intraocular lens.

6. The system of claim 1, wherein the advancement mechanism comprises:
a fluid channel within the handpiece at least partially surrounding the conduit; and
a plunger surrounding the conduit and sealingly disposed within the fluid channel, whereby the plunger is advanceable by pressure within the fluid channel so as to move the lens relative to the insertion tube.

7. The system of claim 1, further comprising a sheath disposed at the distal end of the conduit for containing at least a portion of the lens.

8. The system of claim 1, further comprising a mechanical gripping mechanism disposed at the distal end of the conduit for gripping the lens.

9. The system of claim 8, wherein the gripping mechanism is advanceable and retractable via the handpiece.

10. An intraocular lens insertion and filling system comprising:
a fluidic system including at least one pump and at least one reservoir for a liquid, gas, or solute; and
first and second conduits fluidically connected to the pump and having distal ends configured for (i) contact with an intraocular lens and (ii) cooperation in retaining and filling the lens.

11. The system of claim 10, wherein:
the first conduit extends beyond the second conduit;
a distal end of the first conduit is configured for insertion into the lens; and
the at least one pump is configured to (i) pump liquid from the reservoir through the first conduit and (ii) create a vacuum in the second conduit to retainably draw the lens against a distal end of the second conduit.

12. The system of claim 10, wherein the first and second conduits are concentric.

13. The system of claim 10, wherein the first and second conduits are adjacent.

14. A method of filling an intraocular lens, the method comprising the steps of:
providing a conduit having a distal end disposed within and movable relative to an insertion tube;
inserting the distal end of the conduit into the lens and positioning the lens within the insertion tube;
partially inflating the lens with liquid via the conduit;
causing ejection of the lens from the insertion tube; and
further inflating the lens with the liquid to achieve a target volume.

15. The method of claim 14, wherein the ejection step occurs by mechanically causing relative movement between the insertion tube and the lens therewithin.

16. The method of claim 15, wherein the conduit is advanced relative to the insertion tube.

17. The method of claim 14, wherein the ejection step occurs by fluidically causing relative movement between the insertion tube and the lens therewithin.

18. The method of claim 14, further comprising withdrawing the conduit from the lens following further inflation, whereby the lens has a diameter larger than a diameter of the insertion and is thereby prevented from entry therein.

19. The method of claim 14, wherein the lens is monitored for leakage using at least one of visual detection, optical detection, pressure monitoring, or flow monitoring.

20-43. (canceled)
